(19) United States
(12) Patent Application Publication
(10) Pub. No.: US 2026/0144981 A1
(43) Pub. Date: May 28, 2026

(54) METHODS AND SYSTEMS FOR MONITORING A PURGE SUBSYSTEM OF A CARDIAC SUPPORT SYSTEM

(71) Applicant: Abiomed, Inc., Danvers, MA (US)

(72) Inventors: Iain Zwiebel, Danvers, MA (US); Michelle Graham, Danvers, MA (US); Yuting Zhang, Danvers, MA (US); Samuel Brown, Danvers, MA (US)

(73) Assignee: Abiomed, Inc., Danvers, MA (US)

(21) Appl. No.: 19/354,089

(22) Filed: Oct. 9, 2025

Related U.S. Application Data
(60) Provisional application No. 63/705,723, filed on Oct. 10, 2024.

(51) Int. Cl. A61M 60/829; A61M 60/585

(52) U.S. Cl. CPC A61M 60/829; A61M 60/585; A61M 2205/18; A61M 2205/3334

(57) ABSTRACT

Methods and apparatus for monitoring operation of a purge system of a cardiac support system are provided. The method includes determining, using at least one computer processor, a first baseline value for a purge flow rate signal associated with the purge system based, at least in part, on a purge flow rate signal associated with the purge system and a purge pressure signal associated with the purge system, and outputting via a user interface associated with the cardiac support system an indication of an alarm when a first value of the purge flow rate signal is less than a first threshold value, wherein the first threshold value corresponds to a first percentage of the first baseline value.

### CROSS-REFERENCE TO RELATED APPLICATIONS

This application claims the benefit under 35 U.S.C. § 119(e) to U.S. Provisional Patent Application No. 63/705,723 , filed Oct. 10, 2024, and titled, “METHODS AND SYSTEMS FOR MONITORING A PURGE SUBSYSTEM OF A CARDIAC SUPPORT SYSTEM,” the contents of which is incorporated by reference herein in its entirety.

### FIELD OF THE INVENTION

This disclosure relates to techniques for monitoring a purge subsystem of a cardiac support system that includes an intravascular blood pump.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

An intravascular blood pump may be included as part of a cardiac support system. The cardiac support system may include a purge subsystem configured to prevent ingress of blood into the motor of the blood pump when in operation. A purge fluid may be delivered to an intravascular blood pump assembly via a purge cassette including one or more valves to control the pressure and/or flow rate of the purge fluid in the purge subsystem. Proper functioning of the purge subsystem (e.g., suitable purge flow rates and/or pressure) may be required to preserve blood pump function. Decreased flow rate of purge fluid within the purge subsystem may result from various causes including, but not limited to, partial kinks in the purge tubing or biomaterial buildup within the purge subsystem. In some cases, user interventions may successfully remediate decreases in purge flow rate. However, existing cardiac support systems tend to only alert users when the purge flow rate drops sufficiently to trigger a high purge pressure alarm indicating that the purge subsystem is in a critical state during which the blood pump is already at risk of blood ingress, which may lead to a need to perform patient weaning and/or pump removal to install a new pump. Some embodiments of the present disclosure relate to techniques for monitoring one or more aspects of the purge system to identify potential issues and provide a corresponding alarm prior to the issue representing a critical state.

In one aspect, a method of monitoring operation of a purge system of a cardiac support system is provided. The method includes determining, using at least one computer processor, a first baseline value for a purge flow rate signal associated with the purge system based, at least in part, on a purge flow rate signal associated with the purge system and a purge pressure signal associated with the purge system, and outputting via a user interface associated with the cardiac support system an indication of an alarm when a first value of the purge flow rate signal is less than a first threshold value, wherein the first threshold value corresponds to a first percentage of the first baseline value.

In another aspect, the method further includes determining, when the indication of an alarm is provided via the user interface, whether one or more exit criteria are satisfied, and when the one or more exit criteria are satisfied, removing the indication of the alarm from the user interface. In another aspect, determining whether one or more exit criteria are satisfied includes determining whether a second value of the purge flow rate signal exceeds a second threshold value, wherein the second threshold value is a second percentage of the first baseline value, and determining whether at least one stability criterion associated with the purge flow rate signal and/or the purge pressure signal is met, wherein it is determined that the one or more exit criteria are satisfied when the second value of the purge flow rate signal exceeds the second threshold value and the at least one stability criterion is met. In another aspect, the method further includes determining the second value of the purge flow rate signal as a minimum flow value within an analysis window of the purge flow rate signal. In another aspect, the method further includes decreasing the second threshold value when it is determined that a threshold amount of time has passed since the indication of the alarm was output on the user interface. In another aspect, the second threshold value is higher than the first threshold value. In another aspect, determining whether the at least one stability criterion associated with the purge flow rate signal and/or the purge pressure signal is met includes determining whether a first stability metric associated with the purge flow rate signal is less than a third threshold value, determining whether a second stability metric associated with the purge pressure signal is less than a fourth threshold value, and determining that the at least one stability criterion is met when the first stability metric is less than the third threshold value and the second stability metric is less than the fourth threshold value. In another aspect, the method further includes determining the first stability metric as a coefficient of variation of the purge flow rate signal within a first analysis window of the purge flow rate signal, and determining the second stability metric as a coefficient of variation of the purge pressure signal within the first analysis window of the purge pressure signal. In another aspect, determining the first baseline value comprises determining the first baseline value as a mean flow value within a second analysis window of the purge flow rate signal.

In another aspect, the method further includes determining, using the at least one computer processor, a second baseline value for the purge flow rate signal when the first stability metric is less than the third threshold value and the second stability metric is less than the fourth threshold value, and a threshold amount of time has passed since the indication of the alarm was output via the user interface. In another aspect, determining the second baseline value comprises determining the second baseline value as a mean flow value within the first analysis window of the purge flow rate signal. In another aspect, determining whether one or more exit criteria are satisfied further includes determining whether the second value of the purge flow rate signal is within acceptable operating limits for the purge system, and it is determined that the one or more exit criteria are satisfied when the second value of the purge flow rate signal is within acceptable operating limits for the purge system.

In another aspect, determining the first baseline value includes determining for each of a plurality of time windows of the purge flow rate signal, a corresponding coefficient of variation metric, determining that a first amount of time has passed since the purge flow rate signal was initially received, and determining the first baseline value as a mean flow value within a time window of the plurality of time windows in which the coefficient of variation metric is minimum. In another aspect, the method further includes receiving the purge pressure signal from a pressure sensor associated with the purge system, and calculating the purge flow rate signal based, at least in part, on the purge pressure signal.

In one aspect, cardiac support system is provided. The cardiac support system includes a heart pump including a rotor, a motor configured to drive rotation of the rotor at one or more speeds, a purge system configured to prevent ingress of blood into the motor during operation of the heart pump, the purge system including a pressure sensor, a display configured to provide a user interface, and at least one computer processor. The at least one computer processor is configured to receive a purge pressure signal from the pressure sensor, determine a first baseline value for a purge flow rate signal associated with the purge system based, at least in part, on a purge flow rate signal associated with the purge system and the purge pressure signal, and output via the user interface, an indication of an alarm when a first value of the purge flow rate signal is less than a first threshold value, wherein the first threshold value corresponds to a first percentage of the first baseline value.

In another aspect, the at least one computer processor is further configured to determine, when the indication of an alarm is provided via the user interface, whether one or more exit criteria are satisfied, and when the one or more exit criteria are satisfied, remove the indication of the alarm from the user interface. In another aspect, determining whether one or more exit criteria are satisfied includes determining whether a second value of the purge flow rate signal exceeds a second threshold value, wherein the second threshold value is a second percentage of the first baseline value, and determining whether at least one stability criterion associated with the purge flow rate signal and/or the purge pressure signal is met, wherein it is determined that the one or more exit criteria are satisfied when the second value of the purge flow rate signal exceeds the second threshold value and the at least one stability criterion is met. In another aspect, the at least one computer processor is further configured to determine the second value of the purge flow rate signal as a minimum flow value within an analysis window of the purge flow rate signal. In another aspect, the at least one computer processor is further configured to decrease the second threshold value when it is determined that a threshold amount of time has passed since the indication of the alarm was output on the user interface. In another aspect, the second threshold value is higher than the first threshold value. In another aspect, determining whether the at least one stability criterion associated with the purge flow rate signal and/or the purge pressure signal is met includes determining whether a first stability metric associated with the purge flow rate signal is less than a third threshold value, determining whether a second stability metric associated with the purge pressure signal is less than a fourth threshold value, and determining that the at least one stability criterion is met when the first stability metric is less than the third threshold value and the second stability metric is less than the fourth threshold value. In another aspect, the at least one computer processor is further configured to determine the first stability metric as a coefficient of variation of the purge flow rate signal within a first analysis window of the purge flow rate signal, and determine the second stability metric as a coefficient of variation of the purge pressure signal within the first analysis window of the purge pressure signal. In another aspect, determining the first baseline value comprises determining the first baseline value as a mean flow value within a second analysis window of the purge flow rate signal.

In another aspect, the at least one computer processor is further configured to determine a second baseline value for the purge flow rate signal when the first stability metric is less than the third threshold value and the second stability metric is less than the fourth threshold value, and a threshold amount of time has passed since the indication of the alarm was output via the user interface. In another aspect, determining the second baseline value comprises determining the second baseline value as a mean flow value within the first analysis window of the purge flow rate signal. In another aspect, determining whether one or more exit criteria are satisfied further includes determining whether the second value of the purge flow rate signal is within acceptable operating limits for the purge system, and it is determined that the one or more exit criteria are satisfied when the second value of the purge flow rate signal is within acceptable operating limits for the purge system.

In another aspect, determining the first baseline value includes determining for each of a plurality of analysis windows of the purge flow rate signal, a corresponding coefficient of variation metric, determining that a first amount of time has passed since the purge flow rate signal was initially received, and determining the first baseline value as a mean flow value within an analysis window of the plurality of analysis windows in which the coefficient of variation metric is minimum. In another aspect, the at least one computer processor is further configured to calculate the purge flow rate signal based, at least in part, on the purge pressure signal.

### BRIEF DESCRIPTION OF DRAWINGS

FIG. 1A shows an illustrative cardiac support device that may be used with some embodiments.

FIG. 1B shows an illustrative cardiac support system that includes the cardiac support device of FIG. 1A.

FIG. 2A schematically illustrates a decreasing a purge flow rate signal associated with a purge subsystem of a cardiac support system, in accordance with some embodiments.

FIG. 2B illustrates example purge pressure and purge flow rate signals that may be used to monitor operation of a purge system, in accordance with some embodiments.

FIG. 3 is a flowchart of a process for monitoring operation of a purge system to detect a sustained decreased flow rate, in accordance with some embodiments.

FIG. 4 is a flowchart of a process for determining a baseline value for a purge flow rate signal, in accordance with some embodiments.

FIG. 5 is a flowchart of a process for determining whether exit criteria are satisfied to clear a purge flow rate alarm, in accordance with some embodiments.

FIG. 6 illustrates example scenarios in which a purge flow rate signal recovers to different extents and re-baselining may be performed to establish a new baseline value, in accordance with some embodiments.

FIG. 7 schematically illustrates signals associated with an alarm clearing, in accordance with some embodiments.

### DETAILED DESCRIPTION

Physicians and other healthcare providers may rely on indications of the operational status of a purge subsystem displayed by a controller of a cardiac support device (e.g., an intravascular blood pump) to ensure that the purge subsystem is effectively preventing ingress of blood into the motor of the intravascular blood pump and thereby maintaining normal pump function. The purge subsystem may include one or more sensors (e.g., flow rate sensors, pressure sensors) configured to sense operational aspects of the purge subsystem. Signal(s) sensed by the one or more sensors may be analyzed to detect changes in the signal(s) that may indicate a possible operational issue of the purge subsystem. When such an operational issue is detected, a corresponding alert may be displayed on a user interface associated with the controller to instruct the user that a user intervention to address the operational issue may be needed to maintain normal pump operation.

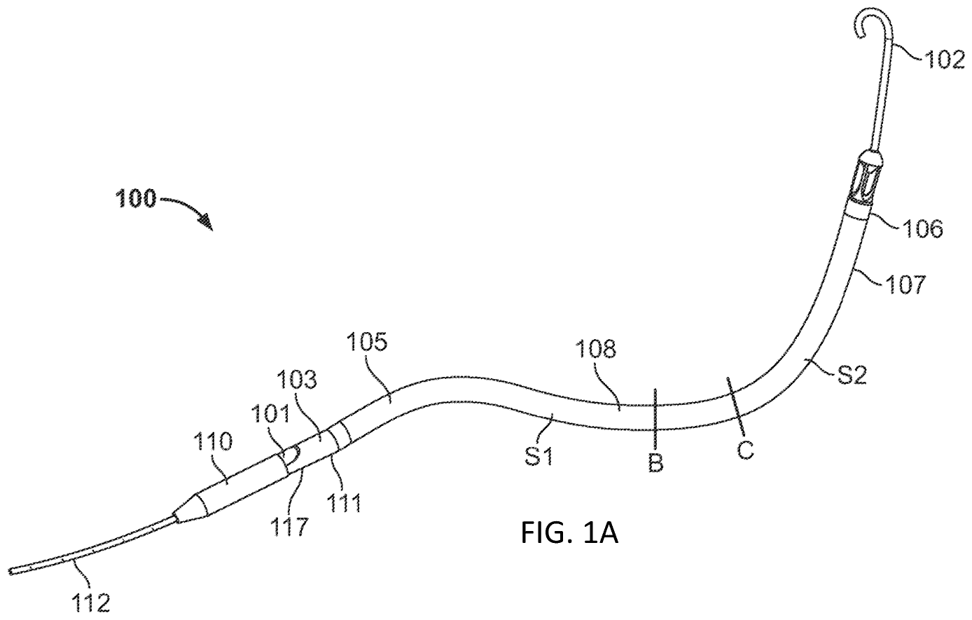

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the wall 111 of the pump housing 103. The pump housing 103 may be composed of a metal in accordance with some implementations. The extension 102, also referred to as a “pigtail,” may be connected to the distal end 107 of the cannula 108 and may assist with stabilizing and/or positioning the blood pump assembly 100 into the correct position in the heart. The pigtail may be configurable from a straight to a partially curved configuration. The pigtail may be composed, at least in part of a flexible material, and may have dual stiffness. It should be appreciated that some embodiments of the pump assembly may not include a pigtail.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or 170°). The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieved the desired shape once inside the heart of the patient.

In some implementations, the blood pump assembly 100 may be inserted percutaneously through the internal jugular vein, through the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta. In some implementations, the blood pump assembly 100 may be inserted percutaneously through the axillary artery across the aortic valve and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid within the purge subsystem 150. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to receive measurements from one or more pressure sensors (not shown) included as a portion of blood pump assembly 100 and purge disc 154. Controller may be configured to determine, based on the pressure sensor measurement(s), a desired flow rate for the purge fluid in the purge subsystem 150 and control operation of a motor to provide the desired flow rate. Controller 130 may also be configured to control operation of the motor (not shown) of the blood pump assembly 100 and purge cassette 153. In some embodiments, controller 130 may be configured to control and measure a pressure and/or flow rate of a purge fluid in purge subsystem 150 via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

As described herein, a purge subsystem (e.g., purge subsystem 150) of a cardiac support system (e.g., cardiac support system 120) may include a pressure sensor configured to sense a purge pressure within the purge subsystem and output a corresponding purge pressure signal. In some embodiments, values of the purge pressure signal may be used in combination with a flow rate control curve to determine a corresponding purge flow rate signal. Some embodiments of the present disclosure include techniques for analyzing the purge pressure signal and/or the purge flow rate signal to detect a potential operational issue (e.g., a sustained decreased flow rate) with the purge subsystem.

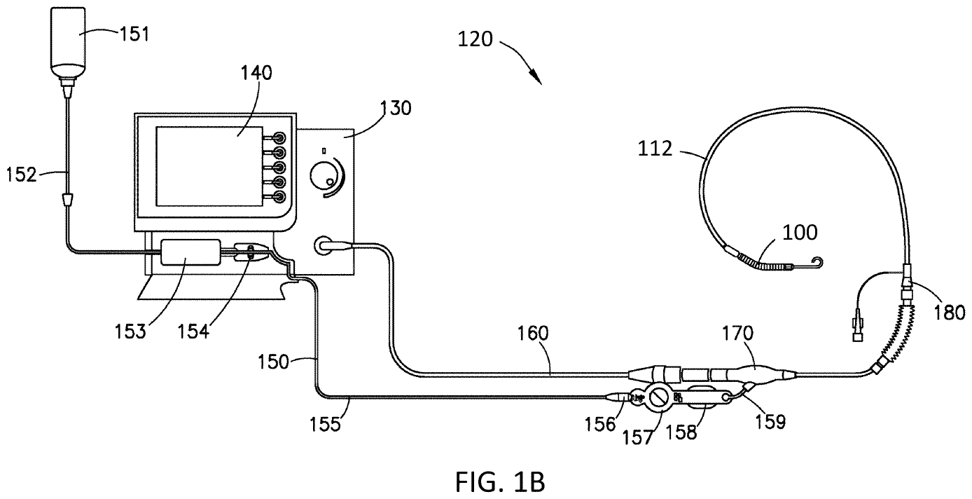

FIG. 2A schematically illustrates a purge flow rate signal output from a purge subsystem, in accordance with some embodiments. As shown in FIG. 2A, the purge flow rate signal may have a relatively stable baseline value (e.g., about 10 mL/hr in the example shown in FIG. 2A) during normal operation. At time T₁, the purge flow starts to gradually decrease and continues to decrease until time T₄ at which point the purge flow rate has fallen below a critical threshold (e.g., 2 mL/hr in the example shown in FIG. 2A) and a high purge pressure alarm is displayed on the controller of the cardiac support system indicating that the cardiac support system is in a critical state. As described herein, the inventors have recognized and appreciated that it may be beneficial to provide an alarm/alert sooner than time T₄, at which point it may no longer be possible to provide a user intervention (e.g., the administration of a drug that breaks down biomaterial in the purge subsystem, bypassing the troublesome section of purge tubing with new tubing, replacing the purge cassette, etc.) that successfully addresses the sustained decrease in purge flow rate without having to remove the pump from the patient. Accordingly, in some embodiments, a sustained decrease in purge flow rate may be detected at time T₂, which is a point in time between T₁ and T₄, and a corresponding alarm may be triggered. As described herein, the sustained decrease in purge flow rate at time T₂ may correspond to a percentage decrease of the baseline value of the flow rate (e.g., at or before time T₁) as observed in the purge flow rate signal. By detecting a sustained decrease in purge flow rate at time T₂, a user intervention may be provided sooner than in previous systems which only alert the user at time T₄. For instance, a drug (e.g., tissue Plasminogen Activator or “tPA”) may be administered within the purge system to breakdown biomaterial that may have formed within the purge subsystem, thereby causing the reduced flow of purge fluid. Administration of the drug may result in recovery of the purge flow rate as indicated by the increasing purge flow rate signal at time T₃. After the purge flow rate has recovered to a sufficient level, the previously triggered alarm may be cleared and normal operation of the cardiac support system may resume. As described herein, in some embodiments, a new baseline value may be established after the purge flow rate has recovered to a sufficient level and further alarms may be triggered with respect to the newly established baseline value.

FIG. 2B illustrates an example purge pressure signal 210 output from a pressure sensor associated with the purge subsystem and an example purge flow rate signal 220 calculated based on the purge pressure signal 210 and a flow rate control curve. For example, as described herein, a purge disc (e.g., purge disc 154) of the purge subsystem may include such a pressure sensor. As shown in FIG. 2B, both the purge pressure signal 210 and the purge flow rate signal 220 may have relatively stable baseline values during normal operation (e.g., prior to time T₁). As also shown, small and/or transient variations in the purge pressure signal and/or the purge flow rate signal during normal operation may occur. For instance, when the pump is turned on or off, the pump speed is changed, the purge bag is changed, or the purge cassette is changed, a temporary drop in purge flow may be observed. In some embodiments, a flow rate threshold value 222 may be set at a percentage of the baseline value established during normal operation (e.g., prior to time T₁). In the example shown in FIG. 2B, the baseline value for the purge flow rate signal 220 may be 10 mL/hr and the flow rate threshold value 222 may be set at 70% of the baseline value (e.g., 7 mL/hr). It should be appreciated however, that the flow rate threshold value 222 may be set at any suitable percentage of the baseline value and 70% is provided merely as one example. For instance, a flow rate threshold value of 60%, 65%, 75% or 80% of the baseline value may alternatively be used. The inventors have recognized that due to manufacturing differences, the baseline value for a purge flow rate signal may vary considerably, while still providing an acceptable amount of purge fluid flow in the purge system. Accordingly, determining the flow rate threshold value 222 as a percentage of the baseline value, in some embodiments, may be used to more accurately identify sustained purge flow rate decreases for a wider range of flow rate baseline values than if the threshold value was set at a fixed amount (e.g., a 2 mL/hr decrease) relative to the baseline value.

At time T₁, the value of the purge flow rate signal 220 has started to decrease and at time T₂ the value of the purge flow rate signal 220 has reached and consistently fallen below the flow rate threshold value 222 for a period of time indicating a sustained decrease in the purge flow rate. As observed in the purge pressure signal, at or around time T₂, the purge pressure signal 210 increases substantially from its baseline value (e.g., prior to time T₁) confirming that an operational issue in the purge system (e.g., a blockage due to biomaterial buildup) exists and user intervention should be attempted to remedy the issue. Following the user intervention, the purge pressure signal 210 and the purge flow rate signal 220 may return to a stable value, albeit perhaps at a different baseline value than was established prior to time T₁.

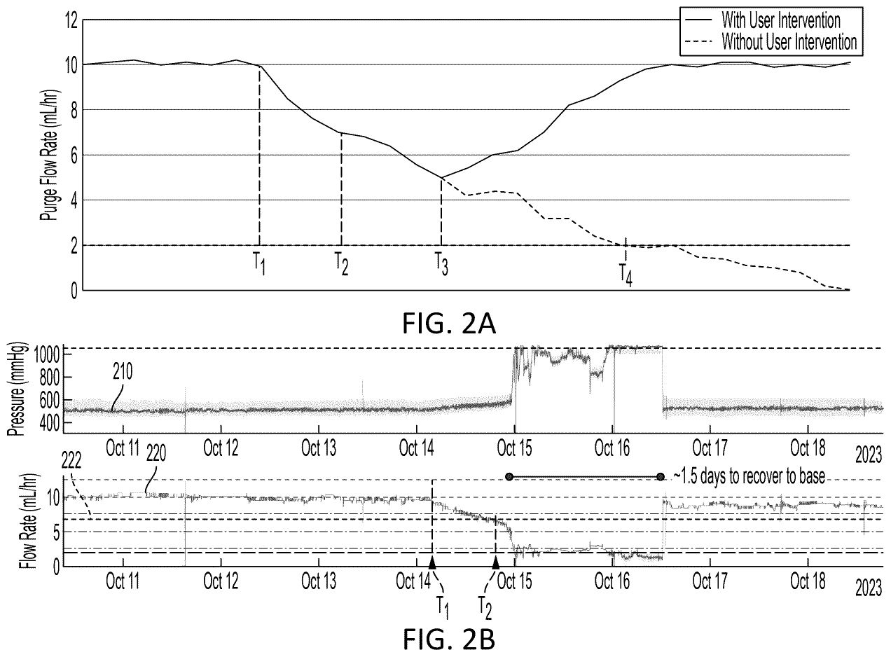

FIG. 3 illustrates a process 300 for detecting a sustained decrease in purge flow rate of a purge system, in accordance with some embodiments. Process 300 may begin in act 310, where an initial baseline value for the purge flow rate is determined. As described herein, manufacturing variances for components of the purge system may result in different baseline purge flow rates when in normal operation. After the purge system has been started, an initial baseline value for the purge flow rate signal may be determined by analyzing values of the purge flow rate signal and/or the purge pressure signal within an analysis window to determine whether the signals are sufficiently stable. In some embodiments, the analysis window may be 20 minutes, 25 minutes, 30 minutes, 35 minutes, 40 minutes, 45 minutes, 1 hour, etc. In some embodiments, determining the initial baseline value comprises calculating a stability metric for each of the purge flow rate signal and the purge pressure signal. As an example, the stability metric may be a coefficient of variation (CV), which may represent a normalized standard deviation (std) within the analysis window

$$( e . g . , CV = \frac{std}{mean} ) .$$

In some embodiments, if the stability metric associated with the purge flow rate signal is less than a first threshold value and the stability metric associated with the purge pressure signal is less than a second threshold value, the initial baseline value may be determined as the mean value of the respective signal within the analysis window.

The inventors have recognized and appreciated that there is a risk that the purge flow rate signal and/or the purge pressure signal may not be stable enough to meet the stability criteria within a reasonable amount of time after the purge system becomes active. In such situations, rather than not setting the initial baseline value, which may result in the purge flow rate alarm never triggering, the initial baseline value may be determined using different criteria after a threshold amount of time has elapsed. For example, a stability metric (e.g., CV) may be determined for the purge pressure signal and the purge flow rate signal during each of a plurality of analysis windows. If the stability metric does not satisfy the stability criteria (e.g., by being less than the respective first and second threshold values) and a particular amount of time (e.g., 3 hours, 4 hours, 5 hours, etc.) has passed since the beginning of the first analysis window, the initial baseline value for the purge flow rate signal may be determined as the mean value of the purge flow rate signal within the analysis window that had the lowest stability metric values (i.e., e.g., the least amount of variation as measured by CV in the analysis window). By setting the initial baseline value in this way, it can be ensured that the purge flow rate alarm calculation described herein will be active after a certain amount of time has passed since the cardiac support system was started, which may be beneficial, for example, to train users of the system with a consistent time to expect the calculation being active.

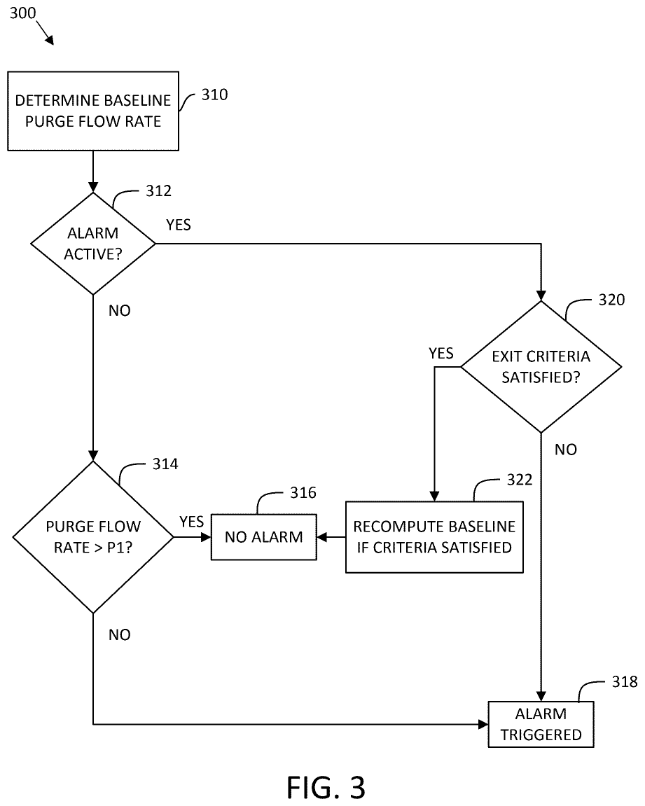

FIG. 4 illustrates a process 400 for assessing stability of purge system signals used to determine a baseline value (e.g., the initial baseline value) for a purge flow rate signal, in accordance with some embodiments of the present disclosure. Process 400 may begin in act 410, where a first stability metric S1 may be determined for the purge flow rate signal. For instance, as described herein, the first stability metric S1 may correspond to a coefficient of variation of the purge flow rate signal within an analysis window. In act 412, a second stability metric S2 may be determined for the purge pressure signal. For instance, as described herein, the second stability metric S2 may correspond to a coefficient of variation of the purge pressure signal within the analysis window. Process 400 may then proceed to acts 414 and 416, where it is determined whether S1 is less than or equal to a third threshold value P3 and whether S2 is less than or equal to a fourth threshold value P4, respectively. If both stability conditions are determined to be satisfied in act 418, process 400 may proceed to act 420, where the baseline value is determined based on the purge flow rate signal. For example, the baseline value may be determined as the mean purge flow rate value within the analysis window used to calculate the first and second stability criteria S1 and S2. If it is determined in act 418 that one or both of the stability criteria are not satisfied, process 400 may proceed to act 422, where it is determined whether a baseline period timeout has expired. For instance, as described herein, if the initial baseline value is not established for more than a threshold amount of time after the purge system is started, an alternate process may be used to establish the initial baseline value such that the alarm can be triggered if the flow rate in the purge system drops. If it is determined that the baseline period timeout has expired (e.g., a threshold amount of time has passed), process 400 may proceed to act 424, where the baseline value is set based on the most stable portion of the purge flow rate history, as described herein. For example, the mean value of the purge flow rate signal within the analysis window associated with the lowest coefficient of variation values may be used to set the initial baseline value.

Returning to FIG. 3, after an initial baseline value for the purge flow rate signal is determined in act 310, process 300 may proceed to act 312, where it is determined whether an indication of the purge flow rate alarm is currently being displayed on a user interface associated with the cardiac support system. If it is determined that the indication of the alarm is not being displayed, process 300 may proceed to act 314, where it is determined whether the purge flow rate signal is greater than a first threshold value P1, which may be a percentage of the current baseline value. For example, as described in reference to FIG. 2B, the value of the purge flow rate signal (e.g., determined as a mean value within a time window) may be compared to a threshold value (e.g., 70% of the baseline value or equivalently a 30% decrease from the baseline value) to determine whether to trigger the purge flow rate alarm. If it is determined in act 314 that the purge flow rate is not greater than the threshold value P1, process 300 may proceed to act 318, where the purge flow rate alarm may be triggered and an indication of the alarm may be output on a user interface associated with the cardiac support system. Alternatively, if it is determined in act 314 that the value of the purge flow rate signal remains greater than the threshold value P1, process 300 may proceed to act 316, where no alarm is triggered. In such an instance, process 300 may return to act 314, where the purge flow rate signal continues to be monitored and compared to the first threshold value P1.

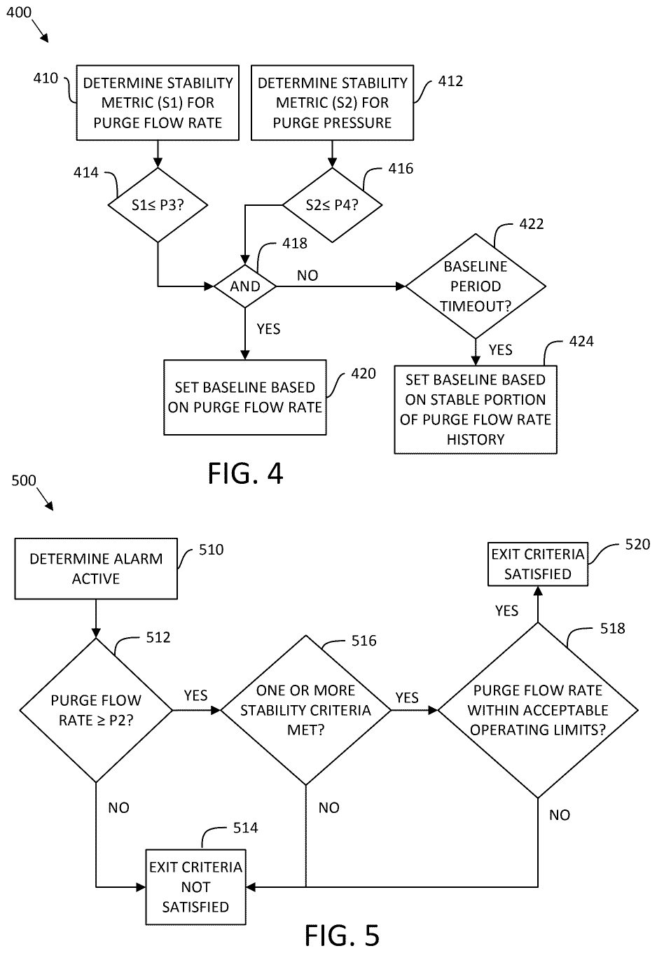

If it is determined in act 312 that the alarm is currently active (e.g., is currently being displayed on the user interface associated with the cardiac support system), process 300 may proceed to act 320, where it is determined whether one or more exit criteria are satisfied and the indication of the alarm can be removed or “cleared” from the user interface. Example exit criteria are discussed in more detail with regard to process 500 shown in FIG. 5. If it is determined in act 320 that the one or more exit criteria are not satisfied, process 300 may proceed to act 318, where the indication of the alarm remains displayed on the user interface. If it is determined in act 320 that the one or more exit criteria are satisfied, process 300 may proceed to act 322 where a new baseline value for the purge flow rate signal (also referred to herein as “re-baselining”) may be determined if one or more re-baselining criteria are satisfied. Example re-baselining criteria are discussed in more detail in process 500 shown in FIG. 5. Regardless of whether re-baselining is performed in act 322, process 300 may proceed to act 316, where the indication of the alarm may be removed from the user interface associated with the cardiac support system. Process 300 may then return to act 314, where the purge flow rate signal continues to be monitored and compared to the threshold value P1, possibly with respect to the new baseline value if re-baselining was performed in act 322.

As described herein, a benefit of alerting a user about a purge system issue earlier than the occurrence of a critical event is that the user may be provided the opportunity to perform a user intervention (e.g., administration of a drug to clear a biomaterial in the purge system, bypassing a troublesome section of purge tubing with new tubing, replacing the purge cassette, etc.) that may enable the purge system of the cardiac support system to recover to a baseline level where normal operation of the blood pump can continue with the indication of the alarm cleared. In some embodiments, determining whether normal operation of the blood pump can continue with the indication of the alarm being cleared is based on one or more exit criteria being satisfied, as discussed in connection with act 320 of process 300.

FIG. 5 illustrates a process 500 for determining whether one or more exit criteria are satisfied, in accordance with some embodiments of the present disclosure. Process 500 may start in act 510 where it is determined that the alarm is active (e.g., by having an indication of the alarm currently displayed on a user interface of the cardiac support system). In some embodiments, determining that one or more exit criteria are satisfied includes determining (1) that the value of the purge flow rate signal has returned to a sufficiently high level, (2) that the recovered purge signals are stable, and (3) that the recovered purge pressure and flow rate signals are below the maximum accepted operating limits of the purge subsystem. To this end, process 500 may proceed to act 512, where it is determined whether the value of the purge flow rate signal is greater than or equal to a second threshold value P2, which may be a percentage of the current baseline value. In some embodiments, the second threshold value P2 may be higher than the first threshold value P1. For instance, if a first threshold value P1 indicated that the purge flow rate signal had to decrease by at least 30% of the baseline value to trigger the alarm, the second threshold value P2 may indicate that the value of the purge flow rate signal may have to increase to a sustained level that is 75% or 80% of the current baseline value to clear the indication of the alarm. It should be appreciated that any suitable value for P1 and P2 may be used, and the threshold values herein are provided merely as examples. It should also be appreciated that the value of the purge flow rate signal and the value of the purge pressure signal may be determined over an analysis window as a mean value, minimum value, or maximum value of the corresponding signal within the analysis window having any suitable length (e.g., 10 minutes, 20 minutes, 30 minutes, etc.) If it is determined in act 512 that the value of the purge flow rate signal has not recovered to a sufficiently high value (e.g., ≥P2), process 500 may proceed to act 514, where it is determined that the exit criteria are not satisfied.

If it is determined in act 512 that the purge flow rate is greater than the second threshold value P2, process 500 may proceed to act 516, where it is determined whether one or more stability criteria are met. As discussed in connection with the process for establishing an initial baseline value, in some embodiments, evaluating the stability of the purge flow rate signal and/or the purge pressure signal may include determining a coefficient of variation (CV) of a respective signal within a time window (e.g., 10 minutes, 20 minutes, 30 minutes, etc.). Accordingly, in some embodiments, determining whether one or more stability criteria are met in act 516 may include (1) determining whether a CV for the purge flow rate signal is greater than a third threshold value P3 and (2) determining whether a CV for the purge pressure signal is greater than a fourth threshold value P4. If it is determined in act 516 that the one or more stability criteria are not met, process 500 may proceed to act 514, where it is determined that the exit criteria are not satisfied.

If it is determined in act 516 that the one or more stability criteria are met, process 500 may proceed to act 518, where it may be determined whether the purge flow rate is within acceptable operating limits. For example, a manufacturer of the purge subsystem may specify that the purge subsystem should operate with a purge flow rate between a lower specification limit (LSL) and an upper specification limit (USL). In act 518, a check may be performed to determine whether the purge flow rate (e.g., the mean purge flow rate within an analysis window) is between the LSL and USL. In one implementation, it may be determined that the purge flow rate is within acceptable operating limits when (1) the mean purge flow rate within an analysis window is less than or equal to the USL and (2) a maximum value of the purge flow rate within the analysis window is greater than a certain percentage (e.g., 70%, 75%, 80%, etc.) of the LSL. If it is determined that the purge flow rate is within acceptable operating limits, process 500 may proceed to act 520, where it is determined that the exit criteria are satisfied. If it is determined that the exit criteria are satisfied, in some embodiments, the baseline value may be recomputed (also referred to herein as “re-baselining”) and the recomputed baseline value may be used for determining if further purge flow rate alarms should be triggered. In some embodiments, the baseline value may be recomputed as the larger value between the mean purge flow rate within the analysis window and the LSL.

The inventors have recognized and appreciated that in situations where the exit criteria are not met (e.g., because the purge flow rate has not recovered to greater than the second threshold value P2) as described herein, it may nonetheless be advantageous to clear the indication of the alarm if an alarm timeout period has elapsed. For example, if the purge flow rate alarm has been displayed on the user interface of the cardiac support system for more than a threshold amount of time (e.g., 1 day, 2 days, 5 days, 1 week, etc.), a less stringent set of exit criteria may be used to clear the indication of the alarm. For example, after a threshold amount of time has elapsed since the indication of the alarm was displayed on the user interface and the value of the purge flow rate signal has not recovered to the second threshold value P2 (also referred to herein as the “clearance threshold”), the clearance threshold may be incrementally lowered (e.g., by 5%, by 10%, etc.) such that it becomes easier to satisfy the clearance threshold portion of the exit criteria. The clearance threshold may continue to be incrementally lowered at fixed timesteps (e.g., every 6 hours, every 12 hours, etc.) until the exit criteria are satisfied in process 500 of FIG. 5, the purge flow level critical threshold (e.g., 2 mL/hr) is reached, after which a different alarm may be provided via the user interface to alert the user to the critical condition that should be addressed, or some other suitable lower limit (e.g., 30% of the current baseline value, 35% of the current baseline value, 40% of the current baseline value, 45% of the current baseline value, 50% of the current baseline value, etc.) is reached. Incrementally relaxing the exit criteria used to clear the purge flow rate alarm, may reduce alarm fatigue (e.g., by having the alarm on frequently and being ignored by the user) and may enable the alarm to re-trigger if the purge flow rate drops again. In some embodiments, when the clearance threshold is incrementally lowered to clear the indication of the alarm, re-baselining may be performed. For example, a new baseline value determined as the mean purge flow rate within the current analysis window for the purge flow rate signal may be used. Incrementally lowering the clearance threshold rather than reducing it substantially in one step may allow for more gradual recovery of the purge flow rate signal prior to establishing a new baseline value.

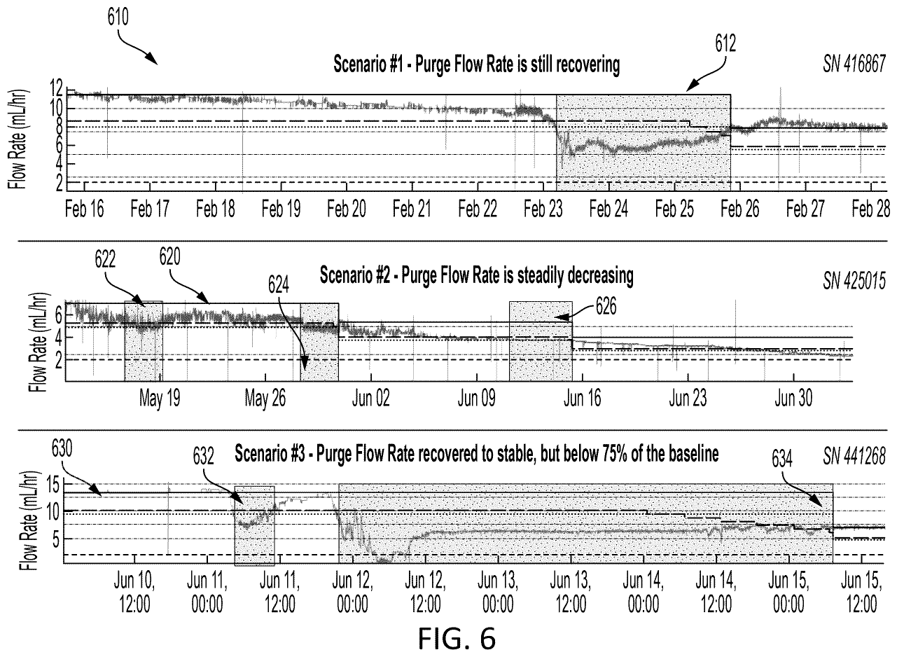

FIG. 6 shows three different scenarios demonstrating a technique for incrementally lowering the clearance threshold, in accordance with some embodiments of the present disclosure. In the first scenario 610, after triggering the purge flow alarm by crossing the first threshold value P1, the purge flow rate signal recovers over the course of three days. Starting at two days post the purge flow rate alarm being triggered, the clearance threshold is incrementally lowered until the value of the purge flow rate signal has recovered sufficiently relative to the clearance threshold. The time window during which the purge flow rate alarm is active is shown as shaded region 612. A new baseline value may then be calculated due to the use of the lowered clearance threshold, and a new first threshold value P1 relative to the new baseline value (e.g., 70% of the new baseline value) may be set. In the second scenario 620, the purge flow rate signal is shown gradually decreasing over a longer timescale compared to scenario 610. In scenario 620, the purge flow alarm is triggered several times corresponding to shaded time windows 622, 624 and 626. As can be observed, because the purge flow rate signal is gradually decreasing over time, re-baselining may be performed after each time the purge flow alarm is cleared. In scenario 630, the purge flow rate alarm is triggered two times corresponding to shaded time windows 632 and 634. Within time window 634, the purge flow rate signal recovers to a stable level that is less than the second threshold value P2 (75% of the current baseline value in the example shown in scenario 630), resulting in establishment of a new baseline value when the alarm is cleared.

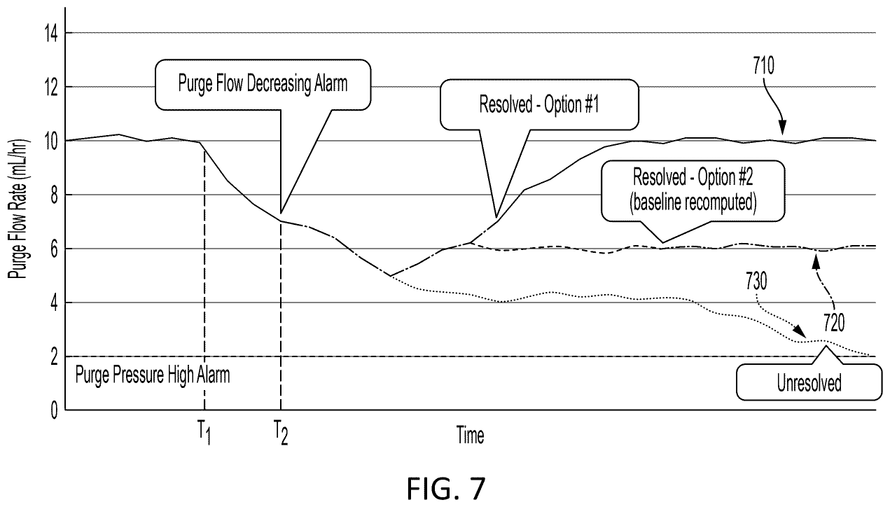

FIG. 7 shows an example plot of purge flow rate vs. time, which illustrates various aspects of alarm clearing, in accordance with some embodiments of the present disclosure. Similar to the plot shown in FIG. 2A, the purge flow rate begins decreasing from a baseline flow rate value at time T₁. At time T₂ the purge flow rate has decreased below a first threshold value P1, which as described herein, may be a percentage of the baseline flow rate value before time T₁. In response to the purge flow rate decreasing below the first threshold value P1 and time T₂, an alarm (e.g., a purge flow decreasing alarm) may be provided to alert a user that a user intervention may be needed to address the decreasing purge flow. Subsequent to the alarm being activated, a user intervention, examples of which are described herein, may be attempted. In response, the purge flow rate may follow one of three trajectories. In a first trajectory 710, the purge flow rate may recover to a level similar to the baseline flow rate value established before time T₁. According to such a first trajectory 710, the alarm may be cleared and the initial baseline flow rate value established before time T₁ may not be recalculated. In a second trajectory 720, the purge flow rate may recover, but to a lower level than the baseline flow rate value established before time T₁. According to such a second trajectory 720, the alarm may be cleared and a new baseline flow rate value may be calculated (e.g., according to one or more of the techniques described herein). In a third trajectory 730, the purge flow rate may continue to decrease indicating that user intervention was not successful in addressing the decreasing purge flow rate and further action (e.g., replacing the pump) may be needed. As shown in FIG. 7, after the purge flow rate falls below a purge pressure threshold value, an alarm indicating the critical state of the purge system may be activated.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of monitoring operation of a purge system of a cardiac support system, the method comprising:
determining, using at least one computer processor, a first baseline value for a purge flow rate signal associated with the purge system based, at least in part, on a purge flow rate signal associated with the purge system and a purge pressure signal associated with the purge system; and
outputting via a user interface associated with the cardiac support system an indication of an alarm when a first value of the purge flow rate signal is less than a first threshold value, wherein the first threshold value corresponds to a first percentage of the first baseline value.

2. The method of claim 1, further comprising:
determining, when the indication of an alarm is provided via the user interface, whether one or more exit criteria are satisfied; and
when the one or more exit criteria are satisfied, removing the indication of the alarm from the user interface.

3. The method of claim 2, wherein determining whether one or more exit criteria are satisfied comprises:
determining whether a second value of the purge flow rate signal exceeds a second threshold value, wherein the second threshold value is a second percentage of the first baseline value; and
determining whether at least one stability criterion associated with the purge flow rate signal and/or the purge pressure signal is met,
wherein it is determined that the one or more exit criteria are satisfied when the second value of the purge flow rate signal exceeds the second threshold value and the at least one stability criterion is met.

4. The method of claim 3, further comprising:
determining the second value of the purge flow rate signal as a minimum flow value within an analysis window of the purge flow rate signal.

5. The method of claim 4, further comprising:
decreasing the second threshold value when it is determined that a threshold amount of time has passed since the indication of the alarm was output on the user interface.

6. The method of claim 3, wherein the second threshold value is higher than the first threshold value.

7. The method of claim 3, wherein determining whether the at least one stability criterion associated with the purge flow rate signal and/or the purge pressure signal is met comprises:
determining whether a first stability metric associated with the purge flow rate signal is less than a third threshold value;
determining whether a second stability metric associated with the purge pressure signal is less than a fourth threshold value; and
determining that the at least one stability criterion is met when the first stability metric is less than the third threshold value and the second stability metric is less than the fourth threshold value.

8. The method of claim 7, further comprising:
determining the first stability metric as a coefficient of variation of the purge flow rate signal within a first analysis window of the purge flow rate signal; and
determining the second stability metric as a coefficient of variation of the purge pressure signal within the first analysis window of the purge pressure signal.

9. The method of claim 8, wherein determining the first baseline value comprises determining the first baseline value as a mean flow value within a second analysis window of the purge flow rate signal.

10. The method of claim 8, further comprising:
determining, using the at least one computer processor, a second baseline value for the purge flow rate signal when:
the first stability metric is less than the third threshold value and the second stability metric is less than the fourth threshold value; and
a threshold amount of time has passed since the indication of the alarm was output via the user interface.

11. The method of claim 10, wherein determining the second baseline value comprises determining the second baseline value as a mean flow value within the first analysis window of the purge flow rate signal.

12. The method of claim 3, wherein determining whether one or more exit criteria are satisfied further comprises:
determining whether the second value of the purge flow rate signal is within acceptable operating limits for the purge system,
wherein it is determined that the one or more exit criteria are satisfied when the second value of the purge flow rate signal is within acceptable operating limits for the purge system.

13. The method of claim 1, wherein determining the first baseline value comprises:
determining for each of a plurality of time windows of the purge flow rate signal, a corresponding coefficient of variation metric;
determining that a first amount of time has passed since the purge flow rate signal was initially received; and
determining the first baseline value as a mean flow value within a time window of the plurality of time windows in which the coefficient of variation metric is minimum.

14. The method of claim 1, further comprising:
receiving the purge pressure signal from a pressure sensor associated with the purge system; and
calculating the purge flow rate signal based, at least in part, on the purge pressure signal.

15. A cardiac support system, comprising:
a heart pump including a rotor;
a motor configured to drive rotation of the rotor at one or more speeds;
a purge system configured to prevent ingress of blood into the motor during operation of the heart pump, the purge system including a pressure sensor;
a display configured to provide a user interface; and
at least one computer processor configured to:
receive a purge pressure signal from the pressure sensor;
determine a first baseline value for a purge flow rate signal associated with the purge system based, at least in part, on a purge flow rate signal associated with the purge system and the purge pressure signal; and
output via the user interface, an indication of an alarm when a first value of the purge flow rate signal is less than a first threshold value, wherein the first threshold value corresponds to a first percentage of the first baseline value.

16. The cardiac support system of claim 15, wherein the at least one computer processor is further configured to:
determine, when the indication of an alarm is provided via the user interface, whether one or more exit criteria are satisfied; and
when the one or more exit criteria are satisfied, remove the indication of the alarm from the user interface.

17. The cardiac support system of claim 16, wherein determining whether one or more exit criteria are satisfied comprises:
determining whether a second value of the purge flow rate signal exceeds a second threshold value, wherein the second threshold value is a second percentage of the first baseline value; and
determining whether at least one stability criterion associated with the purge flow rate signal and/or the purge pressure signal is met,
wherein it is determined that the one or more exit criteria are satisfied when the second value of the purge flow rate signal exceeds the second threshold value and the at least one stability criterion is met.

18. The cardiac support system of claim 17, wherein the at least one computer processor is further configured to:
determine the second value of the purge flow rate signal as a minimum flow value within an analysis window of the purge flow rate signal.

19. The cardiac support system of claim 18, wherein the at least one computer processor is further configured to:
decrease the second threshold value when it is determined that a threshold amount of time has passed since the indication of the alarm was output on the user interface.

20. (canceled)

21. The cardiac support system of claim 17, wherein determining whether the at least one stability criterion associated with the purge flow rate signal and/or the purge pressure signal is met comprises:
determining whether a first stability metric associated with the purge flow rate signal is less than a third threshold value;
determining whether a second stability metric associated with the purge pressure signal is less than a fourth threshold value; and
determining that the at least one stability criterion is met when the first stability metric is less than the third threshold value and the second stability metric is less than the fourth threshold value.

22-28. (canceled)
